(19) United States
(12) Patent Application Publication
(10) Pub. No.: US 2001/0027282 A1
(43) Pub. Date: Oct. 4, 2001

(54) Pain suppression garment

(76) Inventor: Gerald R. Baugh, Portland, OR (US)

Correspondence Address: Kolisch, Hartwell, Dickinson,, McCormack & Heuser, 200 Pacific Building, 520 S.W. Yamhill Street, Portland, OR, 97204, US

(21) Appl. No.: 09/819,132

(22) Filed: Mar. 27, 2001

Related U.S. Application Data
(60) Provisional application No. 60/193,878, filed on Mar. 31, 2000.

(51) Int. Cl. A61F 5/00

(52) U.S. Cl. 602/7; 602/5; 602/19

(57) ABSTRACT

A pain-addressing therapy garment in the form of a wrappable body-expanse which can be fastened to a user's body to apply different selectable levels of compression to the body. A heat generator in the body expanse is activatable to apply heat during use of the garment, and a substance-infused dispenser swathe, also attached to the body expanse, responds to heat to deliver heated moisture (liquid and/or vapor) to the user's body.

### CROSS-REFERENCE TO RELATED APPLICATION

This application claims the priority of U.S. Provisional Patent Application Serial No. 60/193,878 filed on Mar. 31, 2000 and is hereby incorporated by reference.

### BACKGROUND AND SUMMARY OF THE INVENTION

This invention relates to an easily-installed and easily-removed, generally fabric-like heated garment in the form generally of, an elongate band, or band-like element, which can be wrapped around a portion of a person's body (arm, thigh, chest, ankle, neck, wrist, finger, etc.) to deliver heat-based pain-relief. In a preferred embodiment of the invention, the garment of the invention specifically delivers moist-heat (fluid/vapor-heat) relief to the body.

Very generally speaking, the invention, in a preferred embodiment, takes the form of an elongate, wrap-like body expanse of flexible fabric, natural fiber or otherwise, which contains internally distributed heater structure in the form of electrical-resistance wiring that can be energized externally (or internally) by an appropriate source of electrical power to create heat for application to a user's anatomy. Internal power can be battery-supplied, and external power can typically be supplied through suitable adaptive connections provided on the garment for connection, as an illustration, either to an external battery, or to a conventional AC power outlet. An internal battery source can be either replaceable or rechargeable, and can be contained in a suitable pocket or other enclosure formed on/in the garment.

In accordance with the invention, the proposed garment can assume various specific shapes and sizes in order to suit different use applications. For example, some relatively small, elongate, wrap-like garments can be made for a finger or for plural fingers, for the hand, for the foot, etc. Larger wraps can be made for larger regions, say, for the head, the neck, arms, elbows, shoulders, thighs, knees, lower legs, etc. Wraps can also be made to wrap around plural regions in a wearer's anatomy (plural fingers have already been mentioned). Tube-wrap configurations may be made to cover a long stretches of the anatomy, such as a leg. Vest-like wraps are also contemplated. And so on.

A wrap-like garment constructed in accordance with this invention may be constructed either with stretchable or nonstretchable fabric material, and preferably will be made in such a fashion that it can be bound around the anatomy, and be secured adjacent a pain site, with enough flexibility so that the “wrapping pressure” which it exerts on the anatomy is variable/adjustable in accordance with how “tightly” the garment is closed upon itself. While various different closure mechanisms can be employed, one which has great promise for affording such wrapping and pressure flexibility is the conventional hook-and-pile type mechanism that is generally sold under the trademark Velcro.

Resistance wiring, or other suitable, electrically heatable, resistance-type structure, can be suitably distributed in a number of different ways within the body expanse in the garment, and at locations therein selected so that heat can be applied readily to a particular category of anatomical area with respect to which the garment may be especially designed for use. Heating regions can be sectioned for expanded-space or contracted-space heat application, and also to differentiate and enable simultaneously applied, but spatially separated, heat levels.

In a modified form of the invention, non-electrical heating (such as appropriately activated chemical heating) can be employed.

Various embodiments and regions of prospective use for the garment of this invention are described and illustrated generally herein.

Another feature of a preferred embodiment of the garment proposed by this invention involves the concept of equipping that face of the garment which will, effectively, directly face and/or contact the anatomy, with a structure (element), such as a fabric layer or strip (swathe), for carrying an infused, heat-deliverable (either as a liquid, as a vapor, or both) substance, such as a liquid substance like water, or a liquid or gel medicament, or any other kind of selected infusible substance which could, for example, be heat-delivered as a moist vapor to be applied with heat to the anatomy. Such a swathe can readily be made to be removably attachable. It can either be rewashable for repeated use, or it can be made in such a fashion that it can be thrown away as a discardible element. It could also be made as a permanently attached component.

The invention thus offers a therapy, garment-wrap structure designed to apply topical pressure and heat, and most preferably moist heat, effectively and efficiently to and at different specified locations in the human anatomy. The wrap is easily placeable for effective use with selectively different amounts of applied pressure, which pressure cooperates with heat and applied moisture in the ministering to pain.

### BRIEF DESCRIPTION OF THE DRAWINGS

FIG. 1 is a simplified perspective drawing illustrating one embodiment of a therapy-wrap garment structure constructed in accordance with the present invention. A portion of this drawing has been broken away to illustrate details of internal construction, and one component, an infusible moisture-delivering strip component, is shown fragmented in order to simplify the drawing view.

FIGS. 2 and 3 are enlarged, fragmentary, cross-sectional views taken generally along the lines 2-2 and 3-3, respectively, in FIG. 1. These figures show the garment in actual use.

FIG. 4 is an enlarged, fragmentary view taken generally from the region in FIG. 1 which is pointed to by arrows 4-4 in this figure. FIG. 4 depicts certain details of internal construction that further illustrate the garment of FIG. 1. This view further pictures the relationship between heating structure and moisture-delivery structure.

FIG. 5 shows a fragmentary detail of a modified garment having electrical heating structure that is externally energizable.

FIG. 6 shows another modified form of garment constructed in accordance with the invention, and specifically such a garment which employs chemically-created heat.

FIG. 7 is a simplified schematic diagram illustrating still a further modified form of the invention which utilizes electrical wiring to produce heat, and wherein such wiring is organized into separable elements that can be heated, collectively or as individual elements selectively, to apply regional heat to a user's anatomy at differently located topical regions.

FIGS. 8A, 8B and 8C are simplified schematic drawings illustrating three different wrapped conditions which any garment-wrap constructed in accordance with the present invention, such as the garment-wrap of FIG. 1, might assume to apply different levels of therapeutic pressure (compression) to an anatomical region in a user's body, or the fit around differently sized regions.

FIG. 9 is a fragmentary view, on about the same scale as that employed in FIG. 4, showing another modified form of the invention, wherein the garment wrap includes internal electrical heater structure which is segmented, and which is illustrated in two different use applications.

### DETAILED DESCRIPTION OF THE INVENTION

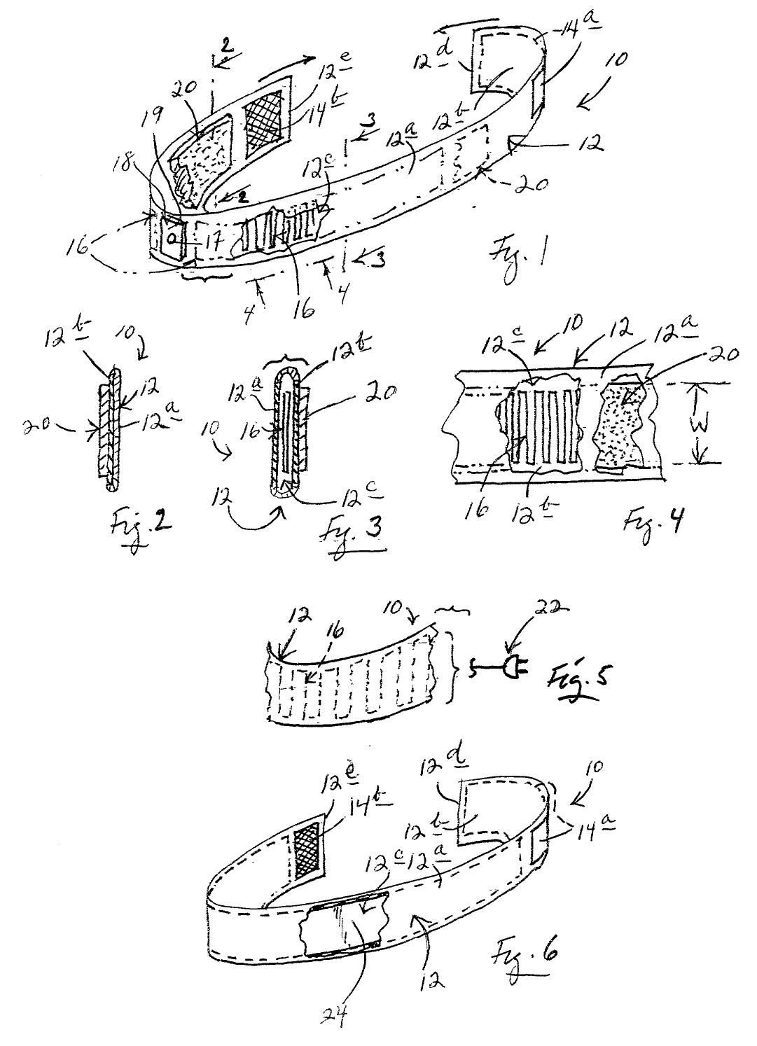

Turning attention now to the drawings, and referring first of all to FIGS. 1-4, inclusive, indicated generally at 10 is one embodiment of a therapy-wrap garment, or garment structure, constructed in accordance with the present invention. As has already been mentioned, the garment of this invention can take a number of different shapes, and can have different appropriate sizes, in order to be used, as will be explained, on and in conjunction with different parts of the anatomy. The garment pictured at 10 in FIGS. 1-4 takes the form generally of an elongate, rectilinear body expanse 12 which might be particularly sized, for example, to wrap around the upper arm of an adult. Body expanse 12, which can be other elastomeric or not so, can be made of any suitable material, such as cotton, nylon, polyester, blends of different natural and/or non-natural materials, or other, and is preferably constructed selectively with a length and a width which is appropriate for the region of the anatomy with respect to which it is intended to be used. Preferably also it is sized to be able to be employed within a selected range of anatomical sizes that might typically characterize such a region. For example, a body expanse length might be chosen to be able to accommodate the upper regions of an adult arm which might lie in a nominal diameter range of about 3- to about 5-inches. The body expanse is constructed with a suitable length so that not only can it be wrapped around such an arm diameter, but also can be secured in a wrapped and end-overlapped condition so as to apply different selected amounts of compression to the arm. Length sizing thus, and for example, is purely a matter of designer choice, and preferably is selected (perhaps about 16-inches in the specific illustration now being discussed) in a manner which allows a given-size garment wrap to be capable of being used throughout such a pre-selected range of application conditions (i.e., anatomical sizes and desired ranges of anatomical compression).

In the embodiment of garment 10 which is now being described, body expanse 12 is formed herein as a two-layer structure including an outer layer 12a and an inner layer 12b suitably assembled to form an elongate, thin, hollow interior chamber 12c.

The opposite ends of expanse 12 are shown at 12d and 12e, and these ends are furnished appropriately with a suitable, adjustable, releasable fastening and closure mechanism, such as components of the well-known product which is sold under the trademark Velcro®. Two Velcro® components 14a, 14b are shown in FIG. 1 appropriately attached to the outside surfaces of layers 12a, 12b, respectively. Components 14a, 14b are located directly adjacent opposite ends 12d, 12e, respectively, in expanse 12. These Velcro® components are appropriately sized so that the body expanse, when garment 10 is in use, can be wrapped in tension around the upper arm region of a user, (within the selected range of arm sizes) and drawn together and fastened by overlapping the ends so as to apply selectively, different amounts of desired compression to the arm. This adjustable compression capability of the invention is one of its advantageous features.

Garment 10 includes, as a heat generator, an electrically energizable, single, elongate heater element 16 which is distributed as wiring, or windings, generally within interior 12c, as shown in cross section in FIG. 3, and in the broken-away portions of body expanse 12 seen in FIGS. 1 and 4. The heater wiring is thus contained in a protected and concealed condition within the body expanse. Generally speaking, heater element 16 is distributed as a kind of continuum throughout a selected, appropriate length of body expanse 12, and with an appropriate and selected width (see W in FIG. 4) relative to the overall width of the body expanse.

The electrical heater element thus present in the garment of FIG. 1 is energized herein selectively by a readily accessible on/off switch 17 which is connected in an appropriate circuit with the heater element and a self-contained battery 18 that is housed in suitable pocket 19 formed in the garment. Battery 18 herein is a non-rechargeable, replaceable battery, though it certainly could, if desired, be a nonremovable, rechargeable battery.

Completing a description now of what is shown in FIGS. 1-4, inclusive, suitably attached, and preferably suitably removably attached, to the outer surface of layer 12b in body expanse 12 is an elongate substance-chargeable (fluid, gel or other solid) fabric strip, or swathe, 20 which is chargeable preferably with a fluid that is to be applied, either in a liquid, vapor or combined form, and under the influence of heat, to an arm region about which garment 10 is wrapped for use. This fabric swathe can be formed of any appropriate material that is suitable for being infused with, and for containing, the to-be-dispensed substance. Preferably, it is formed in such a manner, and/or is mounted in such a manner, that the side of it which faces the outside surface of layer 12b in the body expanse is blocked against fluid leakage from the swathe to the body expanse. Such a desirable fluid barrier can result from construction within the swathe itself, or it can be provided by way of an appropriate barrier expanse incorporated into expanse layer 12b.

Swathe 20, as has been mentioned, is preferably removably attachable to body expanse 12, and is in fact so attached to expanse layer 12b in garment 10, via Velcro® structure which is not specifically illustrated. It should here be noted that swathe 20 in FIG. 1 is shown fragmented so as to simplify this drawing view. A solid-outline fragment portion of the swathe is shown near expanse end 12e, and a separated, dashed-line fragment portion of the swathe is shown near expanse end 12d. It should also be noted here that, while it is preferable that swathe 20 be detachable, for cleaning, charging, etc. purposes, it is not absolutely necessary that it be so, and a garment made in accordance with this invention can indeed be constructed with a permanently attached swathe component.

Focusing specific attention for a moment on FIGS. 2 and 3, and as was mentioned briefly earlier, here, garment 10 is shown (fragmentarily) in a condition of use with the same wrapped around the upper arm portion 22 (also shown fragmentarily) of an adult user's arm. FIG. 2, which is taken from the region indicated by line 2-2 in FIG. 1, illustrates the therapeutic arrangement which thus exists near the overlapping, attached ends of body expanse 12, and very specifically, in the region most nearly adjacent end 12e. FIG. 3 provides a similar showing in relation to a region which is somewhat centrally located between opposite ends of the body expanse. In both illustrations, it is very clear that swathe 20 rests against arm tissue 22, and lies adjacent heater element 16 which is contained between layers 12a, 12b in body expanse 12.

Describing how the garment of this invention can be prepared for use (as illustrated in FIGS. 2 and 3), and focusing first of all on the preferred manner of employing the invention, swathe 20 is suitably charged with a moisture-delivery substance such as, for example, (1) an appropriate fluid medicament, (2) a gel in a similar category, (3) water, or (4) even a substance which is more relatively solid in nature in an unheated condition, but which vaporizes to release moisture under the influence of heat. With charging accomplished, and with the swathe installed in place on the outside of layer 12b, garment 10 is suitably wrapped around the user's arm, and the ends closed upon themselves with releasable attachment effected through the Velcro® components (14a, 14b) in order to achieve a desired level of compression. Obviously, the amount of compression applied is a variable, and is under the control completely of the user. Clearly, the structure of the invention handily accommodates such an adjustable compression behavior.

The user then switches on the heater element, and the garment then, as an overall operative unit, applies compression, heat and moisture to the selected area of the arm which underlies the heater element and the swathe.

When the user has finished with the particular activities just described, the heater element is switched off and the body expanse of the garment is released by uncoupling the opposite ends. In the specific case which has just been described, the fabric swathe is removed and appropriately washed and prepared for reuse in another occasion.

Shifting attention here for a moment to FIGS. 8A, 8B and 8C, and describing the three different garment-use conditions which are illustrated in these three figures, such uses are pictured in these three figures in relation to garment 10 and body expanse 12 as they have so far been described in relation to FIGS. 1-4, inclusive. Accordingly, these same reference numerals are employed in FIGS. 8A, 8B and 8C.

In general terms, these three figures are presented to illustrate in simplified form certain important characteristics of the present invention, as such has so far been described. These characteristics relate to the invention's accommodation for wrapping effectively around anatomical regions of different sizes, and also for accommodating wrapping around a given anatomical region with different levels of body-expanse and overlap so as to produce different levels of wrapped tension, and consequently anatomical compression. Thus, and recognizing the intention that FIGS. 8A, 8B, 8C each illustrate the same garment, in FIG. 8A, the opposite ends 12d, 12e in the body expanse have a relatively modest amount of overlap which produces a tension level T₁ in the wrap for a given arm size that is encircled by the wrap.

In FIG. 8B, body expanse 12 is shown in a condition with a greater amount of overlap between ends 12d and 12e to accommodate a smaller arm than the one “imaginarily pictured” in FIG. 8A, for example, and/or to produce roughly the same amount of arm compression for such a smaller arm, or to produce a greater level of tension (reflected at T₂), and hence, arm compression, in a case where the wrap in FIG. 8B is simply more tightly wrapped around the same arm addressed in FIG. 8A.

FIG. 8C carries further the use condition just described in FIG. 8B. Here, body expanse 12 is shown in an end-overlapped and fastened condition relative to either an arm having a smaller size than the arms addressed in FIG. 8A and FIG. 8B for the same level of compression, or around, for example, the very same arm size addressed in FIG. 8A but with a significantly higher level of body expanse tension T₃, and hence arm compression.

Continuing now with what is shown in the other figures herein, FIG. 5 shows fragmentarily a garment 10 having a body expanse 12 and an internal heating structure, or element, 16 which are much like those same structures pictured, for example, in FIG. 1. A difference here, however, is that the heater structure is intended to be energized externally through appropriate AC-to-DC adaptor circuitry (not shown, but completely conventional in construction), and via a conventional AC wall connection shown generally at 22 in FIG. 5.

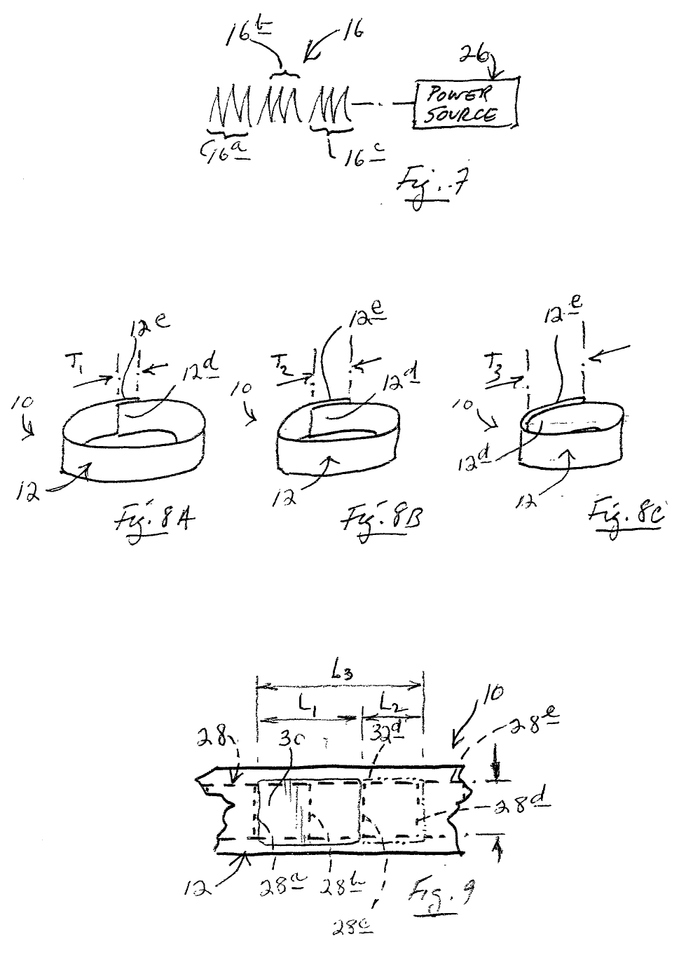

FIG. 6 shows another modified form of a garment wrap 10 which, insofar as the body expanse per se is concerned, is substantially the same in construction as the expanse illustrated and described with reference to FIG. 1. Here, however, the interior region 12c between layers 12a, 12b is furnished with a chemically-activated heater element shown at 24. Element 24 can be of any conventional chemically-activatable heat-component-construction, many of which are known in the art. Such a structure can be inserted into interior 12c through appropriate access openings (not specifically shown, but easily provided appropriately at selected locations) in the structure of expanse 12. Not specifically shown in FIG. 6 in order to simplify the drawing, but present also in the structure of FIG. 6, is a removably attachable (or permanently attached if so desired) moisture-dispensing strip or swathe, like swathe 20 previously described.

FIG. 7 is a fragmentary and schematic diagram showing yet another modified form of the invention wherein an electrical heater element 16 is shown which includes independently energizable heater sections or segments 16a, 16b, 16c. These segments are coupled appropriately to a generically shown power source 26. FIG. 7 thus illustrates an embodiment wherein a garment can be constructed in accordance with the present invention that permits selective energizing of one or more spatially-separated heater elements, thus to control the actual distribution of the regions in an applied garment from which heat is presented. Power source 26 can take the form of any of the kinds of power sources mentioned earlier herein. Another consideration to have in mind is that it is entirely possible to famish a level of control over the energizing of heater segments, such as segments 16a, 16b, 16c, whereby different ones of these elements can be heated to different levels selectively.

FIG. 9 illustrates still another modified form of the therapy garment proposed by the present invention, which garment is again given the reference numeral 10, and which garment includes an elongate body expanse, which is given, once again, the reference numeral 12. In this embodiment of the invention, provided within the interior space between the layers in body expanse 12 is an electrical heater structure 28 which is constructed with segments, such as segments 28a, 28b, 28c, 28d, 28e, that are like previously mentioned segments 16a, 16b, 16c in FIG. 7. These segments can also be energized selectively in the same manner described with respect to the structure of FIG. 7. Additionally illustrated in FIG. 9 is the use of different sizes (and perhaps numbers) moisture-dispensing swathes, such as swathe 30 shown in solid outline in FIG. 9 having a length L₁. Swathe 30 when in place as shown, matchingly overlies heater segments 28b, 28c. In dash-double-dot lines at 32 in FIG. 9, there is shown another size-version of a swathe having a length L₂ which gives it a size allowing it matchingly to overlie but a single heater element, such as heater element 28d. Dimension L₃ in FIG. 9 is employed in this figure to represent yet another-length swathe which might have the collective lengths of L₁ and L₂ to provide yet another specific swathe configuration which would essentially matchingly just overlie the regions covered by heater expanses 28b, 28c, 28d, collectively. Thus, FIG. 9 illustrates yet another region of operational versatility which is offered by the therapy garment of the present invention.

It should thus be apparent that a therapy garment constructed in accordance with the present invention offers a unique structure for ministering to pain through furnishing a highly flexible wrapping garment device for applying heat (preferably moist heat) appropriate medicaments, and different levels of desired compression, to different anatomical regions in the body. For purposes of specific description and illustration herein, several different embodiments of the invention have been shown in the form of generally elongate, flexible, rectilinear structures, with the understanding that the specific shapes of these structures can be adjusted to suit different anatomical uses.

As was mentioned earlier, garments can be made in accordance to the invention (1) to span not only a given anatomical region which may have different effective diametral sizes (i.e. cross-sectional dimensions), thus to apply different amounts of compression to a given anatomical region, (2) to bridge and span around more than a single anatomical element, such as a pair of fingers or so, (3) to be wrapped in a tube-like structure which extends in an elongated fashion along a limb, such as along the length of an arm or a leg, and (4) even to be formed as a more conventional wearable garment, such as a vest, that can be wrapped around the trunk. Heat can be applied utilizing the garment of this invention with such heat furnished variably, electrically or chemically, and with different kinds of power sources, both internal and external furnished for, as an illustration, the electrical applications. Dispensing swathes for promoting the implementation of moist heat application (liquid and/or vapor) can be constructed to contain various different kinds of dispensable substances, and be constructed either to be nonremovable or removable components. Both heating elements and dispensing swathe structures can be shaped and sized to best address different, specific, pain-ministering situations.

Accordingly, while a preferred embodiment and various modifications of the garment of this invention have been illustrated and described herein, it is appreciate that other variations and modifications can be made without departing from the spirit of the invention.

I claim:

1. Garment structure designed for the topical management of pain, via the employment of pressure, heat and moisture (liquid and/or vapor), said structure comprising
an elongate wrap having an operative body expanse usable by the user in relation to a selected anatomical site in the user's body,
a heat generator operatively carried by said wrap, and effective, under a use circumstance, to convey heat to a user's body in the vicinity of said body expanse and a selected anatomical body site,
topical moisture-delivery structure operatively joined/joinable to and with said body expanse in the vicinity of said heat generator, selectively infusible with a substance that can respond to heat to deliver moist heat to a user's body, and
adjustable and releasable closure mechanism, operatively associated with said body expanse, operable to place the body expanse selectively in different levels of tension adjacent a selected anatomical region or regions in a user's body so as to control, selectively, the compression applied by said body expanse to the user's selected anatomical region.

2. The garment structure of claim 1, wherein said wrap is elastomeric.

3. The garment structure of claim 1, wherein said wrap is non-elastomeric.

4. The garment structure of claim 1, wherein said moisture-delivery structure is selectively removable.

5. The garment structure of claim 1, wherein said moisture-delivery structure is non-removable.

6. A wrapper-style pain-therapy garment selectively and removably wearable by a user to cover one or plural anatomical regions of the user's body for the purpose of ministering to pain, said garment comprising
a size-adjustable, selectively tensible, flexible, elongate body expanse adapted generally to encircle a user's selected anatomical region,
adjustable closure structure operatively associated with said body expanse for securing the expanse releasably in a selectively tensed, circumsurrounding, compression-applying manner about a user's selected anatomical region,
a selectively activatable, flexible heat generator joined to said body expanse, adapted to conform with said body expanse under circumstances with the latter wrapped in an operative condition on the anatomy of a user, and
a controlled-delivery, therapeutic, moisture-delivery structure operatively attached, or attachable, to said body expanse at a location adjacent said heat generator, and for selective placement adjacent a user's selected anatomical region with the body expanse wrapped in an operative condition relative thereto,
said moisture-delivery structure being structured to contain, dispensingly, a selected, heat-delivery-promoted moisture (liquid and/or vapor) substance.
